(19) United States
(12) Patent Application Publication
(10) Pub. No.: US 2015/0259723 A1
(43) Pub. Date: Sep. 17, 2015

(54) Firmware Design for Area and Location Data Management of Biological Air Samples Collected on Media Plates

(71) Applicant: Particle Measuring Systems, Inc., Boulder, CO (US)

(72) Inventors: Paul B. HARTIGAN, Longmont, CO (US); Cliff KETCHAM, Golden, CO (US)

(21) Appl. No.: 14/645,853

(22) Filed: Mar. 12, 2015

Related U.S. Application Data
(60) Provisional application No. 61/953,315, filed on Mar. 14, 2014.

(51) Int. Cl. C12Q 1/24; G01N 1/22; C12Q 1/70

(52) U.S. Cl. CPC C12Q 1/24; C12Q 1/70; G01N 1/2226

(57) ABSTRACT

Provided herein are methods and devices that allow for efficient management of many different sampling locations within a facility. A method for operating a biological sampler is described, such as by sampling an environment at a sampling position with the biological sampler and associating the sampling position with a unique identifier, wherein the unique identifier comprises an area and a location. Also provided are associated devices for carrying out the methods.

### CROSS-REFERENCE TO RELATED APPLICATIONS

This application claims the benefit of priority from U.S. Provisional Patent Application No. 61/953,315 filed Mar. 14, 2014, which is hereby incorporated by reference in its entirety to the extent not inconsistent herewith.

### BACKGROUND OF INVENTION

The invention is generally in the field of particle sampling, collection and analysis. The invention relates generally to devices and methods for sampling and characterizing particles in fluids include air and process chemicals (e.g., gases and liquids) for applications including the evaluation of contaminants in a range of cleanroom and manufacturing environments. More specifically, provided are methods and systems that provide for management of many different sampling locations within a facility.

Cleanrooms and clean zones are commonly used in semiconductor and pharmaceutical manufacturing facilities. For the semiconductor industry, an increase in airborne particulate concentration can result in a decrease in fabrication efficiency, as particles that settle on semiconductor wafers will impact or interfere with the small length scale manufacturing processes. For the pharmaceutical industry, where this type of real-time efficiency feedback is lacking, contamination by airborne particulates and biological contaminants puts pharmaceutical products at risk for failing to meet cleanliness level standards established by the Food and Drug Administration (FDA).

Standards for the classification of cleanroom particle levels and standards for testing and monitoring to ensure compliance are provided by ISO 14664-1 and 14664-2. Aerosol optical particle counters are commonly used to determine the airborne particle contamination levels in cleanrooms and clean zones and liquid particle counters are used to optically measure particle contamination levels in process fluids. Where microbiological particles are a particular concern, such as in the pharmaceutical industry, not only is quantification of the number of airborne particles important, but evaluating the viability and identity of microbiological particles is also important. ISO 14698-1 and 14698-2 provide standards for evaluation of cleanroom and clean zone environments for biocontaminants.

Collection and analysis of airborne biological particles is commonly achieved using a variety of techniques including settling plates, contact plates, surface swabbing, fingertip sampling and impactor-based active air samplers. Cascade impactors have traditionally been used for collection and sizing of particles. In these devices, a series of accelerations and inertial impacts successively strip smaller and smaller particles from a fluid flow. Each single stage of an inertial impactor operates on the principle that particles suspended in air can be collected by forcing a dramatic change in the direction of the particle containing airflow, where the inertia of the particle will separate the particle from the airflow streamlines and allow it to impact on the surface. Biswas et al. describe the efficiency at which particles can be collected in a high velocity inertial impactor (Environ. Sci. Technol., 1984, 18(8), 611-616).

In many cleanroom environments, retrieving size information from a particle impactor is not necessary. In this case, a single stage active air sampling impactor system is sufficient to collect biological particle concentrations subject to subsequent detection and analysis. In an impactor-based active air sampler used for collection of biological particles, the impact/collection surface commonly comprises a growth medium, such as an agar plate, as would be used with other biological particle collection techniques. After the particles are collected onto the growth media surface, the media is incubated to allow the biological particles to reproduce. Once the colonies reach a large enough size, they can be identified and characterized, for example using microscopic imaging, fluorescence, staining or other techniques, or simply counted visually by eye or by image analysis techniques.

For these types of biological particle collection and analysis techniques, various operational aspects are important to ensure efficient collection, detection and analysis. For example, the collection efficiency is of critical importance, as failing to detect that biological particles are present in cleanroom air can result in the cleanroom environment having higher levels of contamination than detected. Upon determination that under counting has occurred, pharmaceutical products made in those environments can be identified as failing to meet required standards, potentially leading to costly product recalls. Similarly, failing to ensure that the viability of collected biological particles is maintained during the collection process will also result in under counting. Such a situation can arise, for example, if the collected biological particles are destroyed, damaged or otherwise rendered non-viable upon impact with the growth medium, such that the collected particles do not replicate during the incubation process and, therefore, cannot be subsequently identified.

On the opposite extreme, biological particle concentrations can be overestimated due to false positives. Over counting of this nature arises where a biological particle that is not collected from the cleanroom air, but is otherwise placed in contact with the growth medium, is allowed to replicate during the incubation process and is improperly identified as originating from the cleanroom air. Situations that contribute to false positives include failing to properly sterilize the growth medium and collection system prior to particle collection and improper handling of the growth medium by cleanroom personnel as it is installed into a particle collection system and/or removed from the particle collection system and placed into the incubator. Again, this can result in a pharmaceutical product being identified as failing to meet required standards. Without sufficient measures to identify false positives, such a situation can result in pharmaceutical products that actually meet the required standards, but are destroyed due to an overestimation of biological particle concentration in the cleanroom air indicating that the standards were not met.

There remains a need in the art for particle collection systems capable of achieving efficient sampling of biological particles. For example, particle collection systems are needed for cleanroom and manufacturing applications that provide high particle collection efficiencies while maintaining the viabilities of collected bioparticles. In addition, particle collection systems are needed for cleanroom and manufacturing applications that reduce the occurrence of false positive detection events. There is also a need, particularly for applications requiring a large number of samples, with each sample associated with a specific location in a facility, for managing and tracking of the many different sampling locations within a facility.

### SUMMARY OF THE INVENTION

Provided herein are methods and devices for achieving simple and straightforward management of many different sampling locations within a facility. This management can be particularly challenging for applications where there may hundreds or more of unique sampling locations, and each sampling location having a sample associated therewith.

In an embodiment, the method is for operating a biological sampler by sampling an environment at a sampling position with the biological sampler and associating the sampling position with a unique identifier, wherein the unique identifier comprises an area and a location. Any of the methods, systems and devices provided herein is an integrated method or unit. Such integration is beneficial in terms of sampling management and control, avoiding separate components that must both be moved together and/or connected to each other.

In this manner, a user operating a portable biological sampler may rapidly proceed from sampling position to sampling position taking samples and save time by being able to rapidly access the unique identifier associated with each sampling position, in a rapid, uniform and integrated manner.

For example, the sampling position may be pre-selected and the unique identifier of the sampling position pre-loaded into the biological sampler. This refers to the situation where sampling position is known ahead of time and loaded into the biological sampler. The user of the biological sampler then proceeds to the sampling position and takes the sample.

The methods provided herein, alternatively, are compatible with a user selecting a sampling position and inputting the area and the location of the sampling position into the biological sampler. In this manner, the biological sampler may be considered subsequently pre-set with that input sampling position for later sampling, such as by another user or at a later time and/or date.

In an embodiment, the sampling and associating steps are repeated at a plurality of distinct sampling positions, wherein each sampling position has a unique identifier that is different from a unique identifier of every other sampling position. The methods and devices are compatible with any number of distinct sampling positions. In an aspect, the plurality of distinct sampling positions is greater than or equal to 2 and less than or equal to 1,000.

In an embodiment, the preselected sampling position comprises a plurality of areas, and each area comprises a plurality of locations. In an aspect, the number of areas is selected from a range that is greater than or equal to 2 and less than or equal to 500, and each area is associated with a plurality of locations, wherein the number of locations for each area is independently selected from a range that is greater than or equal to 2 and less than or equal to 500. As the number of sample positions increases, management of associated samples becomes increasingly complicated. The systems and methods provided herein allow rapid selection for sample positions that are associated by area and location. For example, for sample positions that are described as having 10 areas, with each area having 10 locations, selection of an area automatically filters the number of possible sample locations to 10. This is in contrast to conventional samplers where a list of all 100 locations is presented and a user must select one of the 100 locations. This can be a significant resource and time sink with attendant inefficiency. This inefficiency is substantially avoided herein by the association of the sampling position with the unique identifier.

The area and location may correspond to any number of physical locations or descriptors as desired and tailored for the specific application. For example, the area may correspond to a campus, a building, a floor, a process line, or a room. The location may then accordingly correspond to a position within the area. In an aspect, the area corresponds to a room and the location corresponds to a position within the room. In a similar manner, the area may correspond to a process line in a manufacturing application with a first location corresponding to a first sampling position to detect biologicals associated with the process line and a second location corresponding to a second sampling position to detect biologicals in a control location within the process line.

In this manner, as a user enters a room or process line, the area corresponding to the room or process line is provided to the sampler, and the number of possible sample positions accordingly reduced to those having the area associated therewith.

In an aspect, the position is a fixed site within a room.

In an embodiment, the unique identifier comprises at least one additional unique identifier variable that is a sub-location or a supra-area. Such an additional unique identifier variable may be useful to further subdivide the sampling position, such as by floor/room/position; building/room/position; operator/room/position; division/process/position; and the like.

Any of the sampling positions may be labeled to facilitate sampler positioning. The label may be physically observed by a user who can efficiently proceed to the desired position with the sampler. To further improve efficiency, the label may be tagged, wherein the tagging provides automatic identification by the biological sampler of the unique identifier. This may be a label that is bar-coded and read by the sampler, using a radio-frequency identification (RFID) and corresponding reader, or other methods known in the art.

In an embodiment, any of the methods provided herein further comprises the step of identifying the area in which the biological sampler is positioned; and inputting the identified area to the biological sampler data, thereby reducing the number of accessible sampling positions displayed by the biological sampler. In an aspect, the inputting step comprises manual entry by a user of the biological sampler. The inputting step may be further improved by selecting the location from a sampler-displayed list of locations available for the inputted area.

The identifying step may be automated so that a user need not input information directly. In an embodiment, the automated step is selected from the group consisting of: scanning; positioning the sampler in close proximity to a radio frequency identification tag; and tracking a biological sampler position with a positioning receiver connected to the biological sampler. A list of locations associated with the inputted area may be displayed by the biological sampler, and the user can then select from the list.

Any of the methods provided herein may relate to a sampler that has an impact surface for collecting and growing biological particles that have impacted the impact surface. In an embodiment, the sampling comprises exposing an impact surface of the sampler to sample gas; and removing the impact surface from the sampler. As discussed, such sampling that is performed for an individual sampler location becomes difficult to manage when there is a large number of distinct individual sampler locations. The methods provided herein, therefore, are particularly useful for managing such samplers and samples.

In an embodiment, the method further comprises the step of associating the removed impact surface with the unique identifier. In an aspect, the associating the removed impact surface with the unique identifier comprises tagging. The tagging may comprise providing a readable bar code to the impact surface or a container in which the impact surface is confined. In an aspect, the impact surface is an exposed surface of a growth media, such as agar.

Any of the methods provided herein may further comprise the step of observing the growth media for biological growth over a time period and the observing comprises visual detection and/or counting of growth colonies arising from individual viable biological particle impacts with the impact surface.

Any of the methods provided herein may relate to a sampling step that comprises collection of biological particles for a preselected sampling time.

In an embodiment, the method further comprises the step of associating a sample parameter with the unique identifier. Examples of sample parameters include a sample parameter selected from the group consisting of: sampler area; sampler location; a user-provided comment; sample volume; time sampled, sample start date; sample start time; sample end date, sample end time, flow rate; target time; interval; alarms; pauses; an impactor surface serial number; operator identifier; and any combination thereof

In an aspect, the impactor surface is confined within a container such as a petri dish having the impactor surface serial number.

In an embodiment, the method further comprises generating a report comprising at least one impactor parameter.

Any of the methods provided herein may be for a biological sampler to detect biologics in air samples, including viable biologics. The method may be used in an industry selected from the group consisting of: pharmaceutical manufacture, chemical manufacture; food processing; food manufacturing; and bioterrorism detection.

Any of the methods provided herein may further comprise the steps of: selecting an area; and displaying a list of all possible locations associated with the selected area on a graphical user interface connected to the biological sampler. In an embodiment, the graphical user interface is integrated with the biological sampler.

In another embodiment, provided herein is a biological sampler for carrying out any of the methods provided herein. The sampler may comprise a sampling head comprising one or more intake apertures for sampling a fluid flow containing biological particles; an impactor base operationally connected to receive at least a portion of the fluid flow from the sampling head; the impactor base comprising an impact surface for receiving at least a portion of said biological particles in the fluid flow and an outlet for exhausting the fluid flow; a processor for storing one or more sampling positions, wherein the sampling position is associated with a unique identifier comprising an area and a location; and a display operably connected to the processor for displaying all locations associated with an area. The display may comprise a graphical user interface to provide user-selection of one of the locations displayed by the display. In this manner, the sampler position may be rapidly selected during use, thereby minimizing user error and increasing management efficiency, particularly for large number of potential sampling locations.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A and FIG. 1B are schematic illustrations of fluid flow components for use with an impact surface of the sampler and corresponding fluid flow with respect to the impact surface.

FIG. 2 shows a graphical user interface where the area is selected from the main screen.

FIG. 3 shows a graphical user interface that, based on the area selection, displays possible locations associated with that area and provides the ability to create additional locations for the area.

FIG. 4 illustrates a report record generated for the sampling position. As desired, any number of sample parameters may be contained in the report record and the sample parameters may be used with the sample to assist in sample management.

FIG. 5 illustrates an interface for defining unique area/location identifiers along with any other relevant information.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Particle” refers to a small object which is often regarded as a contaminant. A particle can be any material created by the act of friction, for example when two surfaces come into mechanical contact and there is mechanical movement. Particles can be composed of aggregates of material, such as dust, dirt, smoke, ash, water, soot, metal, minerals, or any combination of these or other materials or contaminants. “Particles” may also refer to biological particles, for example, viruses, spores and microorganisms including bacteria, fungi, archaea, protists, other single cell microorganisms and specifically those microorganisms having a size on the order of 1-20 μm. Biological particles include viable biological particles capable of reproduction, for example, upon incubation with a growth media. A particle may refer to any small object which absorbs or scatters light and is thus detectable by an optical particle counter. As used herein, “particle” is intended to be exclusive of the individual atoms or molecules of a carrier fluid, for example, such gases present in air (e.g., oxygen molecules, nitrogen molecules, argon molecule, etc.) or process gases. Some embodiments of the present invention are capable of sampling, collecting, detecting, sizing, and/or counting particles comprising aggregates of material having a size greater than 100 nm, or 10 μm or greater. Specific particles include particles having a size selected from 100 nm to 10 μm or greater.

The expression “sampling a particle” broadly refers to collection of particles in a fluid flow, for example, from an environment undergoing monitoring. Sampling in this context includes transfer of particles in a fluid flow to an impact surface, for example, the receiving surface of a growth medium. Alternatively sampling may refer to passing particles in a fluid through a particle analysis region, for example, for optical detection and/or characterization. Sampling may refer to collection of particles having one or more preselected characteristics, such as size (e.g., cross sectional dimension such as diameter, effective diameter, etc.), particle type (biological or nonbiological, viable or nonviable, etc.) or particle composition. Sampling may optionally include analysis of collected particles, for example, via subsequent optical analysis, imaging analysis or visual analysis. Sampling may optionally include growth of viable biological particles, for example, via an incubation process involving a growth medium. Such growth is a useful indication of viability as well as for assisting in determining presence of biological particles by visual inspection. A sampler refers to a device for sampling particles.

Impactor refers to a device for sampling particles. In some embodiments, an impactor comprises a sample head including one or more intake apertures for sampling a fluid flow containing particles, whereby at least a portion of the particles are directed on to an impact surface for collection, such as the receiving surface of a growth medium (e.g., culture medium such as agar, broth, etc.) or a substrate such as a filter. Impactors of some embodiment, provide a change of direction of the flow after passage through the intake apertures, wherein particles having preselected characteristics (e.g., size greater than a threshold value) do not make the change in direct and, thus, are received by the impact surface. The threshold size value may be selected such as by varying the separation distance between the exit of the intake aperture and the impact surface and/or varying the flow rate through the intake aperture.

The expression “detecting a particle” broadly refers to sensing, identifying the presence of and/or characterizing a particle. In some embodiments, detecting a particle refers to counting particles. In some embodiments, detecting a particle refers to characterizing and/or measuring a physical characteristic of a particle, such as diameter, cross sectional dimension, shape, size, aerodynamic size, or any combination of these. A particle counter is a device for counting the number of particles in a fluid or volume of fluid, and optionally may also provide for characterization of the particles, for example, on the basis of size (e.g., cross sectional dimension such as diameter or effective diameter), particle type (e.g. biological or nonbiological, or particle composition. An optical particle counter is a device that detects particles by measuring scattering, emission or absorbance of light by particles.

“Flow direction” refers to an axis parallel to the direction the bulk of a fluid is moving when a fluid is flowing. For fluid flowing through a straight flow cell, the flow direction is parallel to the path the bulk of the fluid takes. For fluid flowing through a curved flow cell, the flow direction may be considered tangential to the path the bulk of the fluid takes. For laminar flow, flow direction corresponds to the direction of fluid flow streamlines.

“Flow rate” refers to an amount of fluid flowing past a specified point or through a specified area, such as through intake apertures or a fluid outlet of a particle impactor. In one embodiment a flow rate refers to a mass flow rate, i.e., a mass of the fluid flowing past a specified point or through a specified area. In one embodiment a flow rate is a volumetric flow rate, i.e., a volume of the fluid flowing past a specified point or through a specified area. In one embodiment the flow rate may correspond to an average fluid velocity calculated by the volumetric flow rate divided by the cross-sectional area of the fluid conduit in which flow occurs.

Laminar flow refers to a flow that is predictable, steady and not random, in contrast to turbulent flow, and such flows are useful in the devices and methods provided herein to better control impaction of particles satisfying a certain threshold size to improve detection characteristics. Laminar flow refers to flow situations where the ratio of inertial to viscous forces as defined by the Reynolds number (Re=ρVD/μ; ρ is fluid density, V is average velocity, D is a size of the conduit in which the fluid flows, such as aperture dimension or separation distance, and μ is the fluid viscosity), is less than about 2000, less than about 1000, less than about 100, or less than about 1.

“Characteristic dimension” refers to a width, diameter, or effective diameter of a flow channel such as an aperture. Effective diameter corresponds to a diameter for a circle having a cross-section area equivalent to the flow channel or aperture.

“Integrated” or “integrated part” is used herein to refer to any of the methods or systems described herein that is incorporated within a single device. This ensures that the methods are reliably and rapidly performed, within the context of a single platform, without additional external components that must be connected to a central unit. Accordingly, any of the processers, displays and/or inputs, outputs and the like are integrally part of the biological sampler or impactor device. For example, the display may be a touch screen display that a user directly controls and that is an integral part of the impactor device. The associating may occur via a processer that is embedded within or is part of the sampler or device, so that any sampling data is associated with a unique identifier that comprises an area and a location. This is in contrast to embodiments wherein an external device is connected, such as via a hardwire connection or wireless connection, to the sampler device.

### Example 1

### Impactors

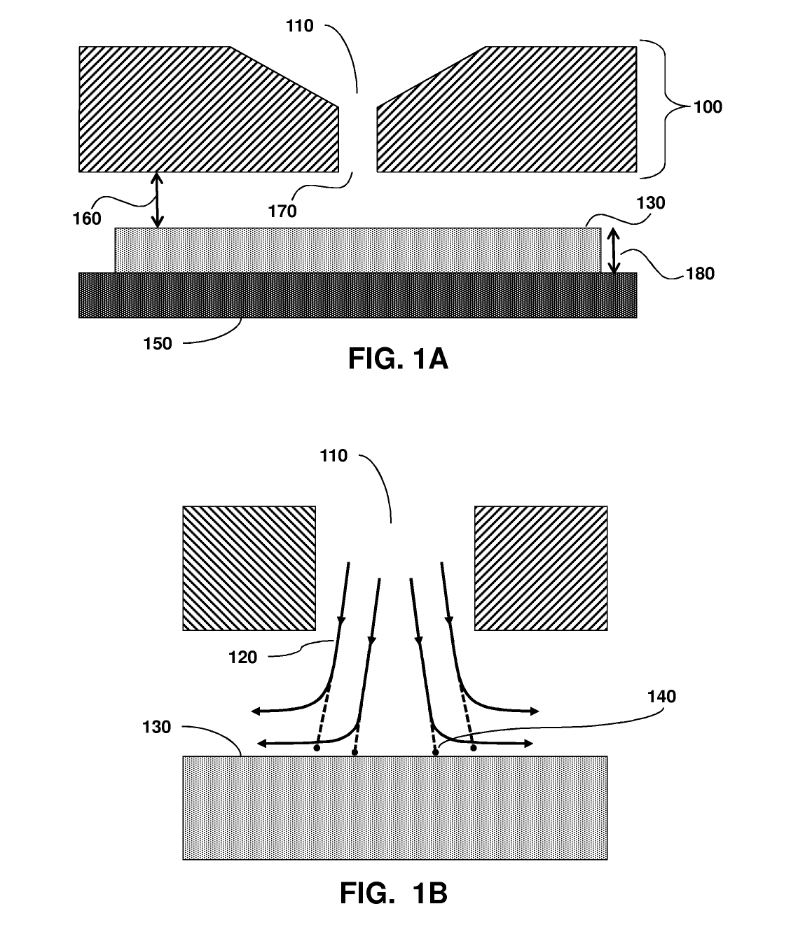

FIG. 1A provides a schematic diagram illustrating the general construction of a particle impactor and FIG. 1B illustrates an expanded view of a particle impactor to further illustrate the operational principal. As shown in these Figures, gas flow is directed through an intake aperture 110 in a sampling head 100 where it is accelerated towards an impact surface 130, which forces the gas to rapidly change direction, following flow paths or streamlines 120 under laminar fluid flow conditions. Due to their momentum, particles 140 entrained in the gas flow are unable to make the rapid change in direction and impact on the impact surface 130. In the embodiment shown in FIG. 1A and FIG. 1B, impact surface 130 is supported by impactor base 150. In embodiments, impact surface 130 comprises the receiving surface of a growth medium, such as agar, provided in a growth medium container or petri dish. Viable biological particles collected on the impact surface, for example, can subsequently be grown and evaluated to provide an analysis of the composition of the fluid flow sampled. For collection of biological particles on the impact surface, control of the separation distance 160, such as a separation distance between the exit 170 of the intake aperture 110 and the impact surface 130, is important. If the distance is too large, for example, the particles may sufficiently follow the fluid path so as to avoid impact with the impact surface. If the distance is too small, however, the particles may impact the impact surface with a force sufficient to render the particles non-viable or otherwise adversely affect the ability of a biological particle to sufficiently reproduce to be visually detected by a user. After sampling, the impact surface is removed and a time period elapsed sufficient for biological particle growth to provide an indication of presence or absence of biological particles. A new impact surface is provided to the sampler for further sampling, such as at another sampling position.

Accordingly, there is a need in the art to manage the sampling, including in view of the potentially very large number of unique sampling positions. Provided herein are methods and devices that assist in sampling management, including by associating each sampling position with a unique identifier. The unique identifier is defined by an area and location tied to the sampling position.

### Example 2

### Firmware Design for Area and Location Data Management of Biological Air Samples Collected on Media Plates

The firmware is structured to allow for simple management of many different sampling locations within a facility.

When samples need to be taken at many locations within a facility the current practice is to either enter a specific location onto a sampler manually every time a sample is taken or to manually track the sample either through the use of external paperwork (or electronic methods), or directly onto the sampling plate.

By creating firmware that structures the samples to be taken into a hierarchal fashion it is possible to identify a specific AREA within a facility for example, Filing Line 1. As well as a specific LOCATION within that area, such as Background Location 1.

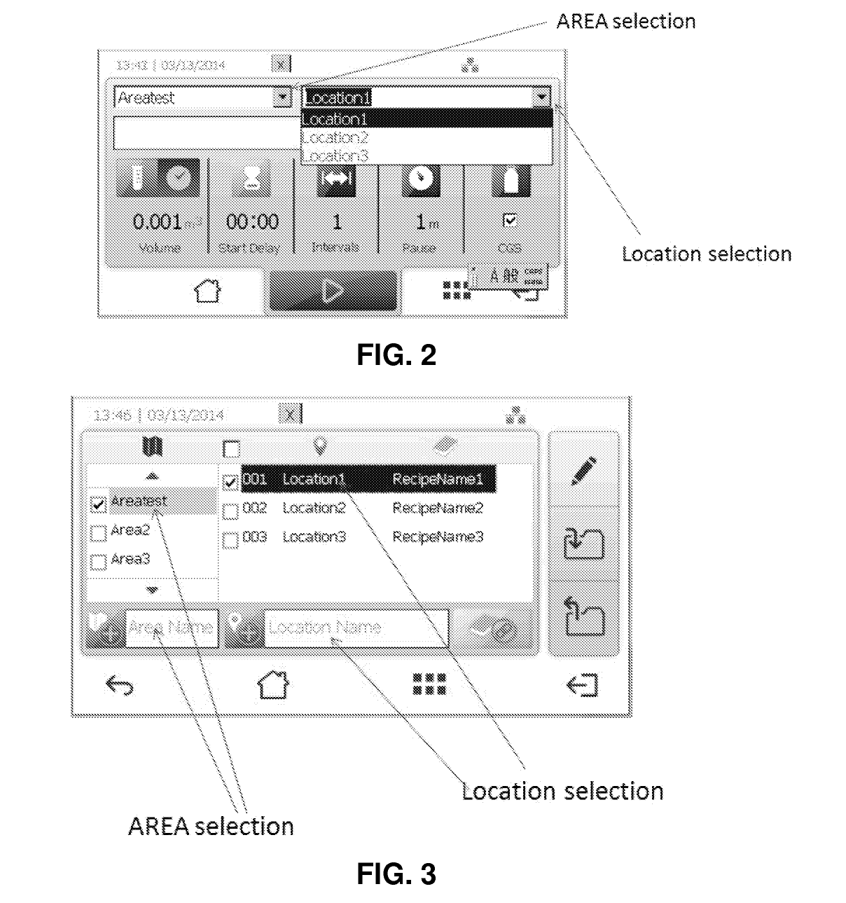

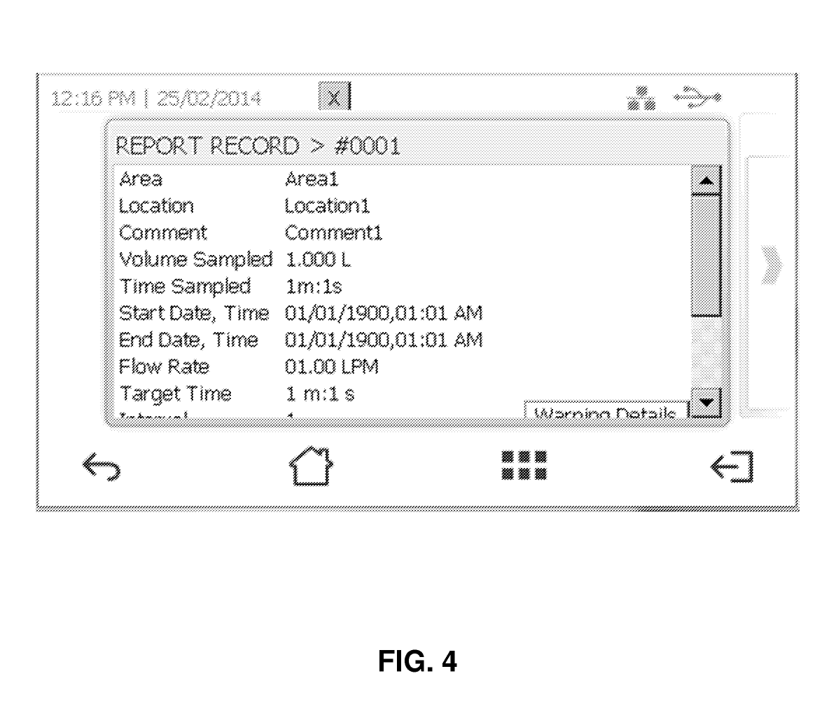

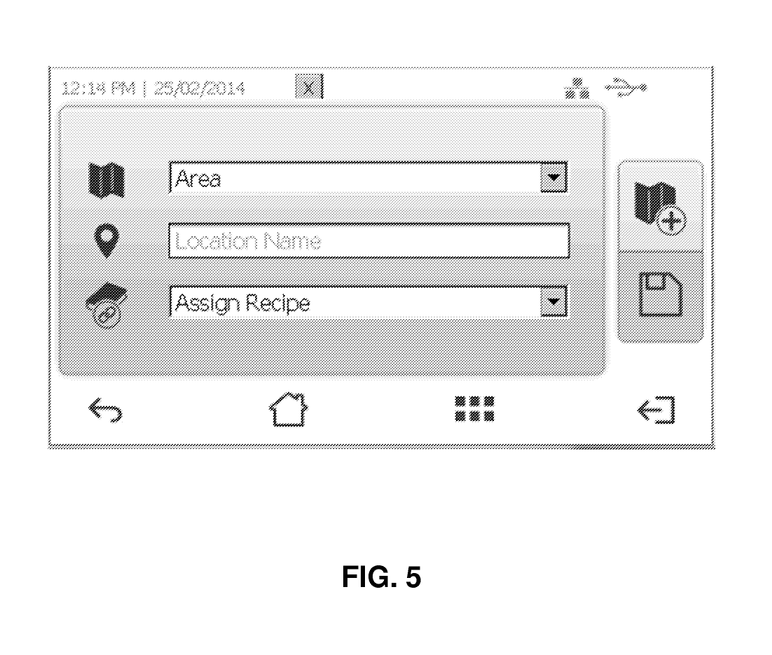

With this type of structure it simplifies the user's selection of the sample point within a particular area and the specific location within that area. This two tiered structure reduces the possibility for error in external recording of the information as well as speeds the ability to identify the proper area and location within that area when taking a sample by having it selectable from a drop down menu. An example is illustrated in FIGS. 2-4, with selection of an area (FIG. 2), corresponding locations associated with that area (FIG. 3), and a generated report record (FIG. 4). FIG. 5 illustrates a user interface to, for example, input a location for a given area and otherwise allow manipulation, variation, and handling of a sampling position.

Existing devices, in contrast, use a single level structure for identification or require manual entry.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for operating a biological sampler, the method comprising the steps of:
sampling an environment at a sampling position with the biological sampler; and
associating the sampling position with a unique identifier, wherein the unique identifier comprises an area and a location, and the associating step is an integral part of the biological sampler.

2. The method of claim 1, wherein the sampling position is pre-selected and the unique identifier of the sampling position pre-loaded into the biological sampler.

3. The method of claim 1, wherein the sampling position is selected by a user of the sampler, the method further comprising the step of:
inputting the area and the location of the sampling position into the biological sampler.

4. The method of claim 1, wherein the sampling and associating steps are repeated at a plurality of distinct sampling positions, wherein each sampling position has a unique identifier that is different from a unique identifier of every other sampling position.

5. The method of claim 4, wherein the plurality of distinct sampling positions is greater than or equal to 2 and less than or equal to 10,000.

6. The method of claim 2 wherein the preselected sampling position comprises a plurality of areas, and each area comprises a plurality of locations.

7. The method of claim 6, wherein the number of areas is selected from a range that is greater than or equal to 2 and less than or equal to 500, and each area is associated with a plurality of locations, wherein the number of locations for each area is independently selected from a range that is greater than or equal to 2 and less than or equal to 500.

8. The method of claim 1, wherein:
the area corresponds to a campus, a building, a floor, a process line, a room; and
the location corresponds to a position within the area.

9. The method of claim 8, wherein the area corresponds to a room and the location corresponds to a position within the room.

10. The method of claim 8, wherein the area corresponds to a process line in a manufacturing application and a first location corresponds to a first sampling position to detect biologicals associated with the process line and a second location corresponds to a second sampling position to detect biologicals in a control location within the process line.

11. The method of claim 8, wherein the position is a fixed site within a room.

12. The method of claim 1, wherein the unique identifier comprises at least one additional unique identifier variable that is a sub-location or a supra-area.

13. The method of claim 1, wherein the sampling position is labeled to facilitate sampler positioning.

14. The method of claim 13, further comprising the step of tagging the label, wherein the tagging provides automatic identification by the biological sampler of the unique identifier.

15. The method of claim 1, further comprising the step of:
identifying the area in which the biological sampler is positioned; and
inputting the identified area to the biological sampler, thereby reducing the number of accessible sampling positions displayed by the biological sampler.

16. The method of claim 15, wherein the inputting step comprises manual entry by a user of the biological sampler.

17. The method of claim 15, further comprising the step of selecting the location from a sampler-displayed list of locations available for the inputted area.

18. The method of claim 15, wherein the identifying step is automated.

19. The method of claim 18, wherein the automated step is selected from the group consisting of:
scanning a label having a scannable element;
positioning the sampler in close proximity to a radio frequency identification tag; and
tracking a biological sampler position with a positioning receiver connected to the biological sampler.

20. The method of claim 19, wherein a list of locations associated with the inputted area is displayed by the biological sampler.

21. The method of claim 1, wherein the sampling comprises:
exposing an impact surface of the sampler to sample gas; and
removing the impact surface from the sampler.

22. The method of claim 21, further comprising the step of associating the removed impact surface with the unique identifier.

23. The method of claim 22, wherein the associating the removed impact surface with the unique identifier comprises tagging.

24. The method of claim 23, wherein the tagging comprises providing a readable bar code to the impact surface.

25. The method of claim 22, wherein the impact surface is an exposed surface of a growth media.

26. The method of claim 25, wherein the growth media comprises agar.

27. The method of claim 25, further comprising the step of observing the growth media for biological growth over a time period.

28. The method of claim 27, wherein the observing comprises visual detection.

29. The method of claim 1, wherein the sampling comprises collection of biological particles for a preselected sampling time.

30. The method of claim 1, further comprising the step of associating a sample parameter with the unique identifier.

31. The method of claim 30, wherein the sample parameter is selected from the group consisting of: sampler area, sampler location, a user-provided comment, sample volume, time sampled, sample start date; sample start time; sample end date, sample end time, flow rate; target time; interval; alarms; pauses, an impactor surface serial number; operator identifier, and any combination thereof

32. The method of claim 31, wherein the impactor surface is confined within a petri dish having the impactor surface serial number.

33. The method of claim 31, further comprising generating a report comprising at least one impactor parameter.

34. The method of claim 1, wherein the biological sampler is for detection of biologics in air samples.

35. The method of claim 34, used in an industry selected from the group consisting of: pharmaceutical manufacture, chemical manufacture; food processing; food manufacturing; bioterrorism detection; tissue banks; cell banks; implant manufacturing; hospitals.

36. The method of claim 1, further comprising the steps of:
selecting an area; and
displaying a list of all possible locations associated with the selected area on a graphical user interface integrated with the biological sampler.

37. A biological sampler comprising:
a sampling head comprising one or more intake apertures for sampling a fluid flow containing biological particles;
an impactor base operationally connected to receive at least a portion of said fluid flow from said sampling head; said impactor base comprising an impact surface for receiving at least a portion of said biological particles in said fluid flow and an outlet for exhausting said fluid flow;
a processor for storing one or more sampling positions, wherein the sampling position is associated with a unique identifier comprising an area and a location; and
a display operably connected to the processor for displaying all locations associated with an area;
wherein the processor and display is an integral part of the biological sampler.

38. The biological sampler of claim 37, wherein the display comprises a graphical user interface to provide user-selection of one of the locations displayed by the display.
